(19) United States
(12) Patent Application Publication
(10) Pub. No.: US 2002/0139378 A1
(43) Pub. Date: Oct. 3, 2002

(54) Method for creating a separation of posterior cortical vitreous from the retina of the eye

(76) Inventors: Michael T. Trese, Bloomfield Hills, MI (US); George A. Williams, Grosse Pointe Park, MI (US)

Correspondence Address: Gifford, Krass, Groh, Sprinkle, Anderson & Citkowski, P.C., Suite 400, 280 N. Old Woodward Avenue, Birmingham, MI, 48009-5394, US

(21) Appl. No.: 09/820,159

(22) Filed: Mar. 28, 2001

(51) Int. Cl. A61B 19/00

(52) U.S. Cl. 128/898; 604/521

(57) ABSTRACT

A method is disclosed for creating a separation of posterior cortical vitreous from the retina of the eye. The method includes the step of introducing plasmin into the vitreous humor of the eye. The plasmin may be introduced either by injection or through a sustained release device. Optionally, other enzymes, polysaccharides, and/or glycoproteins are intermixed with the plasmin.

### BACKGROUND OF THE INVENTION

I. Field of the Invention

The present invention relates generally to medical procedures and, more particularly, to a medical procedure for creating a separation of posterior cortical vitreous from the retina of an eye.

II. Description of Related Art

Certain diseases and/or conditions of the eye, such as diabetes, cystoid macular edema or trauma, produce a vitreoretinal traction on the surface of the retina. If the traction continues, the traction may lead to breaks in the retinal surface and, in severe cases, to retinal detachment.

There have been no previously known treatments for minimizing or eliminating the vitreoretinal traction between the vitreous humor and the retina.

### SUMMARY OF THE PRESENT INVENTION

The present invention provides a method for creating a separation of the posterior cortical vitreous from the retina of the eye and, in doing so, minimize or altogether eliminate the vitreoretinal traction between the vitreous humor and the retina.

The method of the present invention comprises the step of introducing plasmin into the vitreous humor. The introduction of plasmin into the vitreous humor creates a separation of the posterior cortical vitreous and the retina thus minimizing or eliminating the vitreoretinal traction.

The plasmin may be introduced into the vitreous humor either by injection or through a sustained release device. In either event, in order to avoid potentially dangerous effects of increased intraocular pressure, the volume of the plasmin should typically not exceed 0.2 milliliters or cubic centimeters.

Optionally, other enzymes, glycoproteins and/or polysaccharides are intermixed with the plasmin prior to its introduction into the vitreous humor.

### BRIEF DESCRIPTION OF THE DRAWING

A better understanding of the present invention will be had upon reference to the following detailed description, when read in conjunction with the accompanying drawing, wherein like reference characters refer to like parts throughout the several views, and in which:

FIG. 1 is a cross-sectional view of an eye illustrating a first preferred method of the present invention; and

FIG. 2 is a view similar to FIG. 1, but illustrating an alternative method of the present invention.

### DETAILED DESCRIPTION OF PREFERRED EMBODIMENTS OF THE PRESENT INVENTION

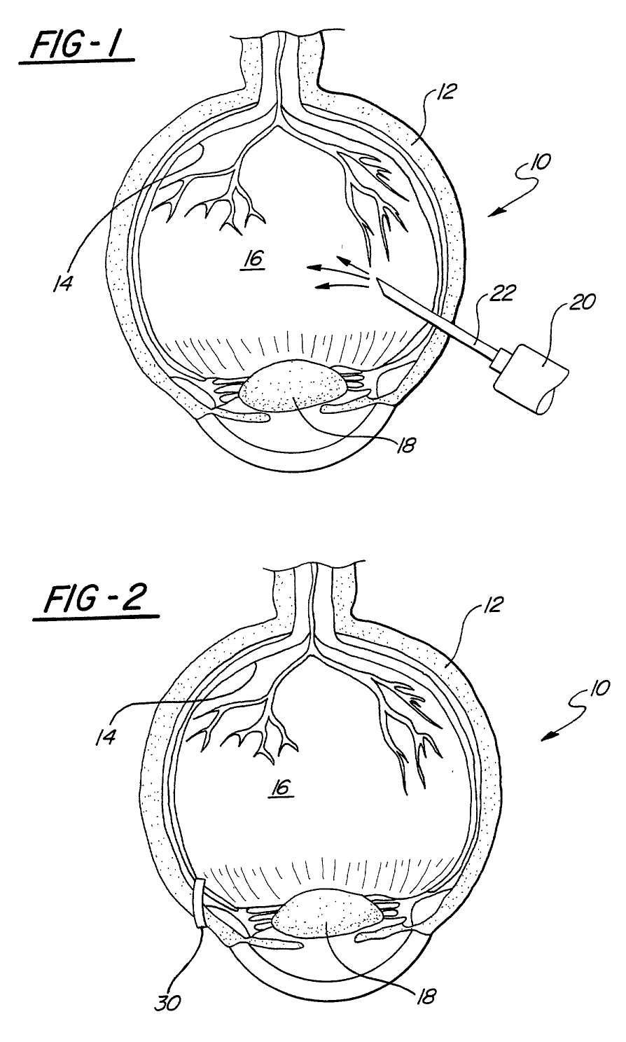

With reference first to FIG. 1, an eye 10, such as a human eye, is there shown in which a sclera forms a generally spherical outer body for the eye 10. A retina 14 extends along the inside reverse surface of the sclera 12 while vitreous humor or vitreous 16 fills the volume of the sclera posterior of the natural eye lens 18.

For persons suffering from certain diseases, most notably diabetes, the vitreous humor 16 adheres more tightly to the retina 14. When this occurs, the adherence produces a vitreoretinal traction between the vitreous 16 and the retina 14. Such traction may lead to breaks or tears in the retina 14 or, in severe cases, to retinal detachment, or macular edema. Other diseases, such as cystoid macular edema and trauma, may produce a similar traction between the vitreous humor 16 and the retina 14.

As shown in FIG. 1, in accordance with the present invention, plasmin 20 is injected by a syringe 22 into the vitreous humor 16. The introduction of the plasmin 20 into the vitreous humor 16 creates a separation of the posterior cortical vitreous from the retina 14 by altering the vitreous molecular structure. This separation, furthermore, minimizes or altogether eliminates the traction between the posterior cortical vitreous and the retina 14 and, in doing so, minimizes or altogether eliminates the possibility of retinal tearing or retinal separation.

In order to prevent potentially dangerous effects of the increased intraocular pressure caused by the introduction of the plasmin 20 into the vitreous 16, preferably no more than 0.2 cubic centimeters of plasmin 20 is introduced into the vitreous humor 16. Alternatively, however, if additional plasmin is necessary to create the desired separation between the vitreous 16 and the retina 14, a portion of the aqueous humor 16 may be removed from the anterior chamber 32 by paracentesis to eliminate excessive intraocular pressure.

Although in the preferred embodiment of the invention, essentially pure plasmin is injected into the vitreous 16, optionally the plasmin may be mixed with other enzymes, glycoprotein and/or polysaccharides. Enzymes operative with plasmin to affect clinically desirable outcomes illustratively include hyaluronidase, chrondroitinase, and collagenase.

With reference now to FIG. 2, an alternate embodiment of the present invention is shown in which a sustained released intraocular device 30, such as that illustratively shown in U.S. Pat. No. 4,135,514, which is incorporated herein by reference, is utilized in lieu of the hypodermic needle 22 to introduce the plasmin into the vitreous 16. As with the first embodiment of the invention, the plasmin may be either essentially pure plasmin or intermixed with glycoproteins and/or polysaccharides.

From the foregoing, it can be seen that the medical procedure of the present invention provides a simple and yet effective means for creating a separation between the posterior cortical surface of the vitreous and the retina of an eye. Having described my invention, however, many modifications thereto will be apparent to those skilled in the art to which it pertains without deviation from the spirit of the invention as defined by the scope of the appended claims.

I claim:

1. A method for creating a separation of posterior cortical vitreous from the retina of an eye consisting of the step of introducing plasmin into the vitreous humor of the eye.

2. The method as defined in claim 1 wherein said introducing step further consists of the step of introducing up to 0.2 cc of plasmin into the vitreous humor.

3. The method as defined in claim 1 wherein said introducing step further consists of the step of introducing a plasmin and glycoprotein mixture into the vitreous humor of the eye.

4. The method as defined in claim 1 wherein said introducing step further consists of the step of introducing plasmin and polysaccharide mixture into the vitreous humor of the eye.

5. The method as defined in claim 1 wherein said introducing step further consists of the step of injecting plasmin into the vitreous humor of the eye.

6. The method as defined in claim 1 wherein said introducing step further consists of the step of using a sustained release device to introduce plasmin into the vitreous humor of the eye.

7. The use of plasmin for the purpose of separating a retina from posterior cortical vitreous within an eye.

8. A vitreoretinal traction therapeutic composition comprising:
plasmin and at least one substance selected from the group consisting of:
hyaluronidase, chondroitinase, and collagenase.
